(19) United States
(12) Patent Application Publication
(10) Pub. No.: US 2014/0120040 A1
(43) Pub. Date: May 1, 2014

(54) ORAL HYGIENE KITS AND METHODS OF PROVIDING SAME

(71) Applicant: Sensible Smiles, Corp., Hanover, ME (US)

(72) Inventor: Joann Moulton, Hanover, ME (US)

(73) Assignee: Sensible Smiles, Corp., Hanover, ME (US)

(21) Appl. No.: 14/063,520

(22) Filed: Oct. 25, 2013

Related U.S. Application Data
(60) Provisional application No. 61/718,813, filed on Oct. 26, 2012.

(51) Int. Cl. A61K 8/96; A61Q 11/00

(52) U.S. Cl. CPC A61K 8/96; A61Q 11/00
USPC 424/48; 424/49

(57) ABSTRACT

Oral hygiene kits and methods of providing same are provided. The oral hygiene kit can include a tooth cleaning device, a food product and a xylitol-containing item selected from the group consisting of a candy, a chewing gum and a combination thereof. A method of reducing tooth decay in a student can include the steps of providing to the student an oral hygiene kit comprising a tooth cleaning device, a food product and a xylitol-containing item selected from the group consisting of a candy, a chewing gum and a combination thereof while the student is at school. In an embodiment, the food product does not have a low pH and/or does not have high fructose corn syrup.

### BACKGROUND

This application is a non-provisional application which claims priority to U.S. Patent Application Ser. No. 61/718,813, filed on Oct. 26, 2012, the entire disclosure of which is incorporated by reference.

The present disclosure relates generally to oral hygiene kits and methods of providing same. More specifically, the present disclosure is directed to a kit comprising a food product, a flossing device, and a xylitol-containing candy and/or chewing gum.

Tooth decay, also known as dental caries, is the number one childhood disease in the United States. See, e.g., Welie, J. V., Justice in Oral Health Care Ethical and Educational Perspectives. Milwaukee: Marquette University Press (2006). Tooth decay is transmissible, infectious, preventable, and curable. Young, D. K., “A Clinician's Guide to CAMBRA,” Compendium, 92-105 (2009).

Tooth decay is multi-factorial. The factors for tooth decay are pathogens in the oral cavity, weaker enamel hosts, and an oral environment that allows for the metabolizing of fermentable carbohydrates. Consuming the wrong foods throughout the day fosters the decay process, and managing the biofilm on teeth is the primary goal of eliminating tooth decay. Teeth are coated with a healthy and an unhealthy film of bacteria in a vehicle of enzymes created from saliva. A low pH in the bacterial colonies causes an imbalance of healthy bacteria to cariogenic bacteria; approximately thirty cariogenic strains of bacteria have been identified in humans.

In patients at risk, the cariogenic bacteria can account for as much as 96% of the biofilm due to a low pH. Kutsch, V. K., “Manage Caries: a minimally invasive approach.” World Congress of Minimally Invasive Dentistry. Imperial Beach, Calif. (2006). The cariogenic bacteria ferment carbohydrates to create an acid that de-mineralizes the surface of the enamel. When the cariogenic bacteria are left unchecked and fed continuously throughout the day, active decay occurs.

A current trend in the United States is to provide healthy alternatives to school children. For example, new guidelines have been legislated for vending machines. (Nixon, “New Guidelines Planned on School Vending Machines,” The New York Times (Feb. 20, 2012)), and the Surgeon General report has dedicated plans for oral health (Surgeon General, Healthy People 2010, retrieved Oct. 2009 from U.S. Department of Human Services: http://www.healthypeople.gov).

Work and school time is lost each year due to untreated tooth decay, and there is an oral health crisis in this country. Many systemic diseases are now attributed to dental disease. As a result, there is a need for improved methods of teaching oral hygiene and products to aid oral hygiene, especially for those who do not have access to a dentist or do not frequently visit the dentist.

### SUMMARY

The present disclosure provides oral hygiene kits and methods of providing same. In an embodiment, an oral hygiene kit comprising a tooth cleaning device, a food product and a xylitol-containing item selected from the group consisting of a candy, a chewing gum and a combination thereof is provided.

In an embodiment, the tooth cleaning device is selected from the group consisting of dental floss, a toothbrush, a toothpick, an inter-proximal brush and combinations thereof.

In an embodiment, the food product is selected from the group consisting of vegetables, nuts, seeds, cheeses, meats, popcorns, yogurts, eggs, seafood and combinations thereof.

In an embodiment, at least one of the food product or the xylitol-containing item can comprise probiotics.

In an embodiment, the xylitol-containing item comprises a chewing gum and a candy.

In an embodiment, the oral hygiene kit further comprises a bottle of water.

In an embodiment, the food product does not comprise high fructose corn syrup.

In an embodiment, the food product has a pH of at least 5.0.

In an embodiment, the food product has a pH of at least 6.0.

In an embodiment, the food product has a pH of at least 7.0.

In an embodiment, the food product has a pH of at least 8.0.

In an embodiment, the food product has a pH of at least 9.0.

In another embodiment, a method of reducing tooth decay in a student is provided. The method comprises the steps of providing to the student an oral hygiene kit comprising a tooth cleaning device, a food product and a xylitol-containing item selected from the group consisting of a candy, a chewing gum and a combination thereof while the student is at school.

In an embodiment, the oral hygiene kit is provided to the student by a school employee.

In an embodiment, the oral hygiene kit is provided to the student by a vending machine.

In another embodiment, a method of promoting oral hygiene in an individual in need thereof is provided. The method comprises the steps of providing to the individual an oral hygiene kit comprising a tooth cleaning device, a food product and a xylitol-containing item selected from the group consisting of a candy, a chewing gum and a combination thereof.

In another embodiment, a method of preventing dental caries in an individual in need thereof is provided. The method comprises the steps of administering a food product and a xylitol-containing item selected from the group consisting of a candy, a chewing gum and a combination thereof to the individual and providing a tooth cleaning device substantially concurrently.

In another embodiment, a method of promoting oral hygiene is provided. The method comprises the steps of substantially concurrently consuming a food product having a characteristic selected from the group consisting of a pH of at least 5.0, not having high fructose corn syrup, and a combination thereof; using a tooth cleaning device; and using a xylitol-containing item selected from the group consisting of a candy, a chewing gum and a combination thereof.

In an embodiment, the food product has a pH of at least 6.0.

In an embodiment, the food product has a pH of at least 7.0.

In an embodiment, the food product has a pH of at least 8.0.

In an embodiment, the food product has a pH of at least 9.0.

An advantage of the present disclosure is to provide an oral hygiene kit.

Another advantage of the present disclosure is to provide methods of providing an oral hygiene kit.

Still another advantage of the present disclosure is to reduce the incidence of dental caries.

Yet another advantage of the present disclosure is to provide oral hygiene kits having a chewing gum and/or a candy that comprise xylitol.

Another advantage of the present disclosure is to combine the need to combine a food product with an implement to clean teeth and a chewing gum and/or a candy designed to reduce the bacterial load directly following consumption of the food product.

Still another advantage of the present disclosure is to provide a kit having a food product selected based on frequency of consumption, form of the food product, pH of the food product, and amount and type of carbohydrates in the food product.

Yet another advantage of the present disclosure is to combine food that cannot be metabolized into sugar with an implement for cleaning between the teeth and a chewing gum or candy that kills pathogenic bacteria.

Another advantage of the present disclosure is to provide an oral hygiene kit available in stores and/or vending machines.

Still another advantage of the present disclosure is to provide an oral hygiene kit to children while at school.

Yet another advantage of the present disclosure is to manage the biofilm on teeth.

Additional features and advantages are described herein, and will be apparent from the following Detailed Description and the figures.

### BRIEF DESCRIPTION OF THE FIGURES

FIG. 1 shows a perspective view of an oral hygiene kit in accordance with an embodiment of the present disclosure.

FIG. 2 shows a perspective view of an oral hygiene kit in accordance with an embodiment of the present disclosure.

### DETAILED DESCRIPTION

All ranges contained within this application are intended to include all numbers, whole or fractions, contained within said range. As used in this disclosure and the appended claims, the singular forms “a,” “an” and “the” include plural referents unless the context clearly dictates otherwise. “Substantially concurrent” means actions performed in time periods that are within thirty minutes of each other, preferably within fifteen minutes of each other, more preferably within five minutes of each other, and most preferably at the same time.

The present disclosure is related to oral hygiene kits and methods of providing oral hygiene kits. As used herein, a “kit” means that the identified components are physically associated in or with one or more containers and considered a unit for manufacture, distribution, sale, or use. Containers include, but are not limited to, bags, boxes, cartons, bottles, packages of any type or design or material, over-wrap, shrink-wrap, affixed components (e.g., stapled, adhered, or the like), or combinations thereof. A single package may be one or more containers that contain the identified components, and the one or more containers are physically associated such that they are considered a unit for manufacture, distribution, sale or use.

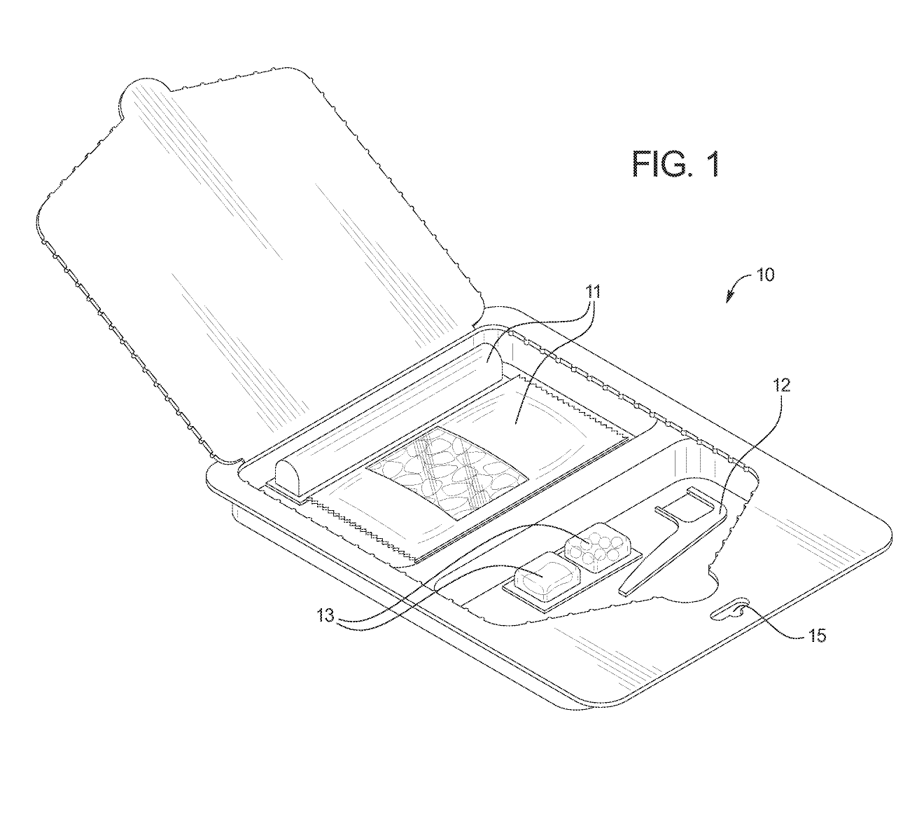

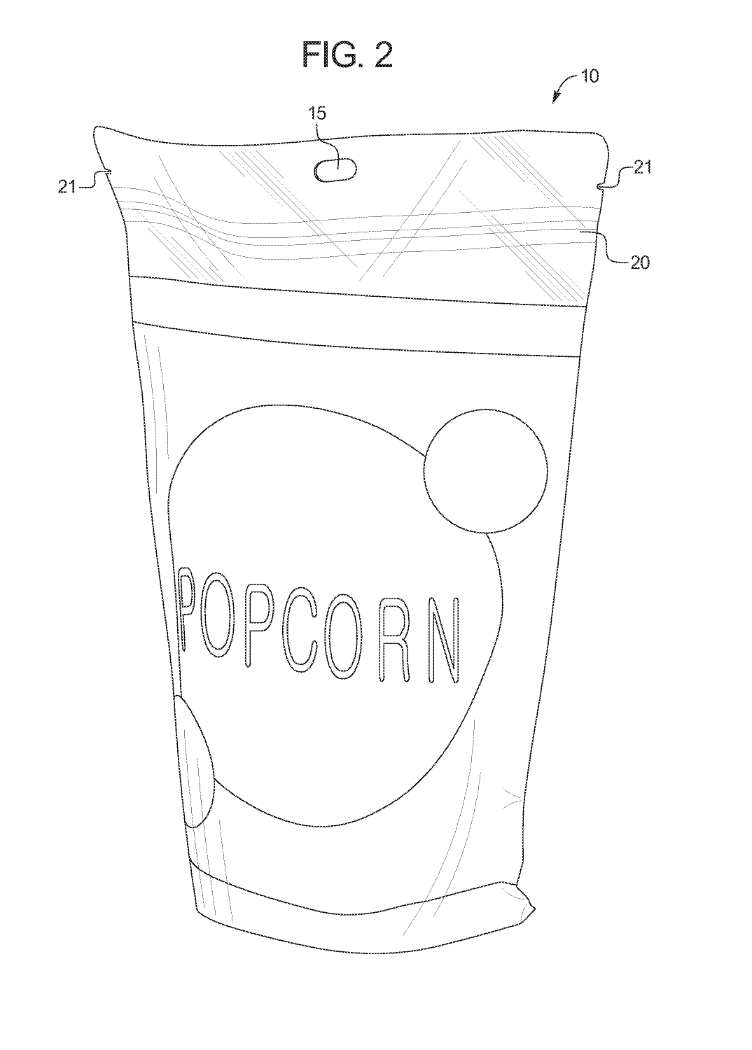

FIGS. 1 and 2 generally illustrate embodiments of an oral hygiene kit 10 according to the present disclosure. The oral hygiene kit 10 can be accessed using any means known to the skilled artisan, such as a perforated opening, a re-sealable opening, a removable lid or the like. For example, as shown in FIG. 2, the oral hygiene kit 10 can comprise a re-sealable zipper 20 which can open and close, and the re-sealable zipper 20 can be accessed by tearing a portion of the oral hygiene kit 10, such as by using a tear notch 21 in the oral hygiene kit 10.

Referring again to FIGS. 1 and 2, the oral hygiene kit 10 comprises a food product 11, a cleaning device 12 and a xylitol-containing candy and/or chewing gum 13 (hereafter “the xylitol gum 13”). The xylitol gum 13 can comprise one or more pieces of an item that is a chewing gum, a candy or a combination thereof. Furthermore, the xylitol gum 13 can comprise one or more pieces of candy and one or more pieces of chewing gum. For example, in a non-limiting embodiment depicted in FIG. 1, the xylitol gum 13 comprises several small pieces of hard candy and a single piece of chewing gum. The xylitol gum 13 is not limited to a specific embodiment, and the xylitol gum 13 may comprise any candy and/or chewing gum comprising xylitol known to the skilled artisan.

The xylitol gum 13 comprises xylitol, which has the chemical formula of (CHOH)₃(CH₂OH)₂. The xylitol gum 13 can comprise other ingredients, such as elastomers, plasticizers, resins, fillers, antioxidants, colorants and/or flavorants such as other sweeteners additional to the xylitol. In an embodiment, the xylitol gum 13 is contained in one or more containers that separate the xylitol gum 13 from the food product 11 and/or the cleaning device 12. For example, the food product 11, the cleaning device 12 and the xylitol gum 13 can be packaged together in a single package while the xylitol gum 13 is packed in at least one other container within the single package. The xylitol gum 13 can be consumed directly following consumption of the food product 11 or use of the cleaning device 12.

The cleaning device 12 can be any device that can be used for tooth cleaning. For example, the cleaning device 12 can be a spool of dental floss; a disposable toothbrush comprising dry toothpaste applied to the bristles to be activated by water or saliva; a toothpick; an inter-proximal pick with a tapered cone for insertion between teeth; a device that discharges a stream of water, an inter-proximal brush with fine, pointed bristles; and/or the like. In an embodiment generally depicted in FIG. 1, the cleaning device 12 comprises a dental floss holder having prongs with dental floss therebetween, such as an “F” flosser. Alternatively or additionally, the cleaning device 12 can comprise a “Y” flosser. Dental floss may be, for example, unwaxed floss, waxed floss, polytetrafluoro-ethylene floss, dental tape and/or the like. In an embodiment, the cleaning device 12 can be used by individuals having orthodontic braces. The cleaning device 12 is not limited to a specific embodiment, and the cleaning device 12 may be any tooth cleaning device known to the skilled artisan.

The food product 11 is preferably selected based on frequency of consumption, form of the food product 11, pH of the food product 11, and/or amount and type of carbohydrates in the food product 11. In an embodiment, the food product 11 does not have a low pH and/or does not have high fructose corn syrup. For example, the food product 11 can have a pH that is at least 5.0, more preferably at least 6.0, even more preferably at least 7.0, still even more preferably at least 8.0, and most preferably at least 9.0. However, the food product 11 is not limited to a specific pH. Preferably, any sugar in the food product 11 is naturally present and has not been added to the food product 11.

The food product 11 can be a meal and/or can be intended to be consumed as a snack e.g. between meals. The food product 11 can comprise, for example, vegetables such as carrot sticks or celery; nuts such as peanuts, cashews, almonds, pistachio nuts, mixed nuts or the like; seeds such as pumpkin seeds, sunflower seeds or pepitas; peanut butter; cheese such as a block of cheese, a cheese dip, a slice, or a stick and example flavors being Colby-Jack, Swiss, American, Cheddar, Provolone, Cream and the like; jerky such as beef jerky or turkey jerky; popcorn, such as plain popcorn or cheese popcorn; yogurt; tuna with or without mayonnaise; deli meats; egg salad; meats such as turkey, chicken, pork, beef, ham or pepperoni; or seafood such as salmon, sardines, shrimp, crabmeat, lobster, oysters or clams; or combinations thereof. In an embodiment, the meat may be pre-cooked.

A preferred combination is cheese and a meat such as jerky. In an embodiment depicted in FIG. 1, the food product 11 comprises cheese and seeds. The food product 11 is not limited to a specific embodiment, and the food product 11 may be any food product, and preferably any food product not having a low pH and/or not having high fructose corn syrup, known to the skilled artisan.

The food product 11 and/or the xylitol gum 13 can comprise probiotics. As defined by the World Health Organization, probiotics are live microorganisms which when administered in adequate amounts confer a health benefit on the host. In an embodiment, the xylitol gum 13 can comprise a probiotic-containing chewing gum.

The kit 10 can also comprise a bottle of water. In an embodiment, the kit 10 and/or a container within the kit 10 comprise indicia providing instructions and/or information, such as nutrition information for the food product 11 and/or the xylitol gum 13, instructions for using the cleaning device 12, or the like. For example, the kit 10 and/or a container within the kit 10, such as a container for the xylitol gum 13, can indicate that individuals with braces should not use chewing gum.

Without wishing to be bound by any theory, the inventors believe that the oral hygiene kit 10 reduces the incidence of dental caries by addressing the need to snack at school with an implement to clean teeth, a product designed to reduce the bacterial load directly following the snack, and a food product selected as previously set forth. Specifically, food that cannot be metabolized into sugar prevents tooth decay because cariogenic bacteria metabolize sugar and also adhere to glycoproteins on the teeth that are formed by sugars. In addition, xylitol kills cariogenic bacteria that attempt to metabolize it. Moreover, the kit provides all of these items at one time to the individual, such as a student at a school, to encourage their substantially concurrent use.

As a result, the present disclosure provides an oral hygiene kit comprising a tooth cleaning device, a food product and a xylitol-containing item selected from the group consisting of a candy, a chewing gum and a combination thereof. The tooth cleaning device can be selected from the group consisting of dental floss, a device having prongs with dental floss therebetween, a toothbrush, a toothpick, an inter-proximal brush, an inter-proximal pick, a device that discharges a stream of water, and combinations thereof. The food product can be selected from the group consisting of vegetables, nuts, seeds, cheeses, meats, popcorns, yogurts, eggs, seafood and combinations thereof. In an embodiment, at least one of the food product or the xylitol-containing item can comprise probiotics. The xylitol-containing item can comprise a chewing gum and a candy. In an embodiment, the oral hygiene kit further comprises a bottle of water.

In an embodiment, the food product does not comprise high fructose corn syrup. In some embodiments, the food product has a pH of at least 5.0, at least 6.0, at least 7.0, at least 8.0 or at least 9.0.

Another aspect of the present disclosure provides methods for providing the oral hygiene kit 10. For example, the oral hygiene kit 10 can have a hole 15 that enables hanging of the oral hygiene kit 10 in retail environments and/or vending machines. Vending machines can refrigerate the oral hygiene kit 10 to prevent the food product from spoiling. In an embodiment, the oral hygiene kit 10 is provided to students at a school. For example, the school can have a vending machine that comprises the oral hygiene kit 10, and/or a school employee such as a teacher, a cafeteria worker or the like can provide the oral hygiene kit 10. For example, the student can be 21 years old or younger, 18 years old or younger, 15 years old or younger, or 12 years old or younger. The oral hygiene kit 10 can be provided at lunchtime. Alternatively or additionally, the oral hygiene kit 10 can be provided so that the food product 11 is consumed as a snack; for example, the oral hygiene kit 10 can be provided between meals.

As a result, the present disclosure provides a method of reducing tooth decay in a student, the method comprising the steps of providing to the student an oral hygiene kit comprising a tooth cleaning device, a food product and a xylitol-containing item selected from the group consisting of a candy, a chewing gum and a combination thereof while the student is at school. The oral hygiene kit can be provided to the student by a school employee and/or a vending machine.

The present disclosure also provides a method of promoting oral hygiene in an individual in need thereof, the method comprising the steps of providing to the individual an oral hygiene kit comprising a tooth cleaning device, a food product and a xylitol-containing item selected from the group consisting of a candy, a chewing gum and a combination thereof. In addition, the present disclosure provides a method of preventing dental caries in an individual in need thereof, the method comprising the steps of administering a food product and a xylitol-containing item selected from the group consisting of a candy, a chewing gum and a combination thereof to the individual and providing a tooth cleaning device substantially concurrently.

Moreover, the present disclosure provides a method of promoting oral hygiene, the method comprising the substantially concurrent steps of consuming a food product having a characteristic selected from the group consisting of a pH of at least 5.0, not having high fructose corn syrup, and a combination thereof; using a tooth cleaning device; and using a xylitol-containing item selected from the group consisting of a candy, a chewing gum and a combination thereof. In some embodiments, the food product has a pH of at least 5.0, at least 6.0, at least 7.0, at least 8.0 or at least 9.0.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. An oral hygiene kit comprising a tooth cleaning device, a food product and a xylitol-containing item selected from the group consisting of a candy, a chewing gum and a combination thereof.

2. The oral hygiene kit of claim 1 wherein the tooth cleaning device is selected from the group consisting of dental floss, a device having prongs with dental floss therebetween, a toothbrush, a toothpick, an inter-proximal brush, an inter-proximal pick, a device that discharges a stream of water, and combinations thereof.

3. The oral hygiene kit of claim 1 wherein the food product is selected from the group consisting of vegetables, nuts, seeds, cheeses, meats, popcorns, yogurts, eggs, seafood and combinations thereof.

4. The oral hygiene kit of claim 1 wherein at least one of the food product or the xylitol-containing item comprises probiotics.

5. The oral hygiene kit of claim 1 wherein the xylitol-containing item comprises a chewing gum and a candy.

6. The oral hygiene kit of claim 1 further comprising a bottle of water.

7. The oral hygiene kit of claim 1 wherein the food product does not comprise high fructose corn syrup.

8. The oral hygiene kit of claim 1 wherein the food product has a pH of at least 5.0.

9. The oral hygiene kit of claim 1 wherein the food product has a pH of at least 6.0.

10. The oral hygiene kit of claim 1 wherein the food product has a pH of at least 7.0.

11. The oral hygiene kit of claim 1 wherein the food product has a pH of at least 8.0.

12. The oral hygiene kit of claim 1 wherein the food product has a pH of at least 9.0.

13. A method of reducing tooth decay in a student, the method comprising the steps of providing to the student an oral hygiene kit comprising a tooth cleaning device, a food product and a xylitol-containing item selected from the group consisting of a candy, a chewing gum and a combination thereof while the student is at school.

14. The method of claim 12 wherein the oral hygiene kit is provided to the student by a school employee.

15. The method of claim 12 wherein the oral hygiene kit is provided to the student by a vending machine.

16. A method of promoting oral hygiene in an individual in need thereof, the method comprising the steps of providing to the individual an oral hygiene kit comprising a tooth cleaning device, a food product and a xylitol-containing item selected from the group consisting of a candy, a chewing gum and a combination thereof.

17. A method of preventing dental caries in an individual in need thereof, the method comprising the steps of administering a food product and a xylitol-containing item selected from the group consisting of a candy, a chewing gum and a combination thereof to the individual and providing a tooth cleaning device substantially concurrently.

18. A method of promoting oral hygiene, the method comprising the substantially concurrent steps of consuming a food product having a characteristic selected from the group consisting of a pH of at least 5.0, not having high fructose corn syrup, and a combination thereof;
using a tooth cleaning device; and using a xylitol-containing item selected from the group consisting of a candy, a chewing gum and a combination thereof.

19. The method of claim 18 wherein the food product has a pH of at least 6.0.

20. The method of claim 18 wherein the food product has a pH of at least 7.0.

21. The method of claim 18 wherein the food product has a pH of at least 8.0.

22. The method of claim 18 wherein the food product has a pH of at least 9.0.
